(19) United States
(12) Patent Application Publication
(10) Pub. No.: US 2026/0115454 A1
(43) Pub. Date: Apr. 30, 2026

(54) CATHETER BLOOD PUMPS AND ASSOCIATED METHODS

(71) Applicant: Supira Medical, Inc., Los Gatos, CA (US)

(72) Inventor: Daniel HILDEBRAND, Santa Cruz, CA (US)

(21) Appl. No.: 19/479,307

(22) PCT Filed: Apr. 25, 2024
(86) PCT No.: PCT/US2024/026313

§ 371 Date: Oct. 27, 2025

Related U.S. Application Data
(60) Provisional application No. 63/498,137, filed on Apr. 25, 2023.
(60) Provisional application No. 63/580,833, filed on Sep. 6, 2023.

(51) Int. Cl. A61M 60/867; A61M 60/13; A61M 60/226; A61M 60/403; A61M 60/554

(52) U.S. Cl. CPC A61M 60/867; A61M 60/13; A61M 60/226; A61M 60/403; A61M 60/554

(57) ABSTRACT

Catheter blood pump positioning techniques, methods, and algorithms are described. In one embodiment, pressure sensor measurements are used to determine the distance to the aortic valve and the left ventricle. An optimal positioning location can be determined based on the pressure sensor measurements. The catheter blood pump can then be optimally positioned to locate the inlet in the ventricle and the outlet in the aorta.

WO

### PRIORITY CLAIM

This patent application claims priority to U.S. Provisional Ser. No. 63/498,137, titled “CATHETER BLOOD PUMPS AND ASSOCIATED METHODS,” and filed on Apr. 25, 2023, and U.S. Provisional Ser. No. 63/580,833, titled “CATHETER BLOOD PUMPS AND ASSOCIATED METHODS,” and filed on Sep. 6, 2023, which are both herein incorporated by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Patients with heart disease can have severely compromised ability to drive blood flow through the heart and vasculature, presenting for example substantial risks during corrective procedures such as balloon angioplasty and stent delivery. Intra-aortic balloon pumps (IABP) are used to support circulatory function, such as treating heart failure patients. An IABP is typically placed within the aorta, and inflated and deflated in counter-pulsation fashion with the heart contractions, with one function being to provide additive support to the circulatory system. Use of IABPs is common for treatment of heart failure patients, such as supporting a patient during high-risk percutaneous coronary intervention (HRPCI), stabilizing patient blood flow after cardiogenic shock, treating a patient associated with acute myocardial infarction (AMI) or treating decompensated heart failure. Such circulatory support may be used alone or in with pharmacological treatment.

More recently, minimally invasive rotary blood pumps have been developed, which are inserted into the body in connection with the cardiovascular system to pump arterial blood from the left ventricle into the aorta to add to the native blood pumping ability of the left side of the patient's heart. Another known method is to pump venous blood from the right ventricle to the pulmonary artery to add to the native blood pumping ability of the right side of the patient's heart. An overall goal is to reduce the workload on the patient's heart muscle to stabilize the patient, such as during a medical procedure that may put additional stress on the heart, to stabilize the patient prior to heart transplant, or for continuing support of the patient. The smallest rotary blood pumps currently available may be percutaneously inserted into the vasculature of a patient through an access sheath, thereby avoiding more extensive surgical intervention, or through a vascular access graft. One such device is a percutaneously inserted ventricular support device.

Although blood pumps exist, there is a need to provide additional improvements in the field of ventricular support devices and similar blood pumps for treating compromised cardiac blood flow.

### SUMMARY OF THE DISCLOSURE

The disclosure is related to intravascular blood pump and methods of their use.

A method of positioning a catheter blood pump is provided, comprising: advancing a catheter blood pump across an aortic valve of a subject; continuously or intermittently recording pressure signals in the heart with a pressure sensor of the catheter blood pump; stopping advancement of the catheter blood pump when a left ventricular pressure signal is detected with the pressure sensor; recording a first distance from a fixed location on, in, or outside the subject to a left ventricle, DLV; pulling back the catheter blood pump into the aorta until an aortic pressure signal is detected with the pressure sensor; recording a second distance from the fixed location to the aortic valve, DAO; determining a target distance to advance the catheter blood pump based on the first distance, the second distance, and catheter blood pump dimensions; and advancing the catheter blood pump by the target distance to optimally position the pump.

In some aspects, the pressure sensor is positioned near a distal end of the catheter blood pump.

In one aspect, the pressure sensor is positioned near a proximal end of the catheter blood pump.

In another aspect, the catheter blood pump is optimally positioned with an inlet in the ventricle and an outlet in the aorta.

In some aspects, an impeller of the catheter blood pump is positioned in the aorta.

In one embodiment, determining the target distance comprises solving for DTarget-DS+(DLV−DAO)/2, where DTarget-DS is the distance from DTarget, the targeted location, to the pressure sensor.

A method of positioning a catheter blood pump is also provided, comprising: advancing a catheter blood pump across an aortic valve of a subject; continuously or intermittently recording pressure signals in the heart with a distal pressure sensor and a proximal pressure sensor of the catheter blood pump; recording a first distance from a fixed location on, in, or outside the subject to a left ventricle, DLV, when a first left ventricular pressure signal is detected with the distal pressure sensor; recording a second distance from a fixed location on, in, or outside the subject to the left ventricle, DLV, when a second left ventricular pressure signal is detected with the proximal pressure sensor; determining a target distance to pull back the catheter blood pump based on the first distance, the second distance, and catheter blood pump dimensions; and pulling back the catheter blood pump by the target distance to optimally position the pump.

A catheter blood pump, comprising: an expandable blood conduit defining a lumen between an inlet and an outlet; an impeller at least partially disposed within the blood conduit; a proximal pressure sensor positioned proximally from the outlet; a distal pressure sensor positioned distally from the inlet; an elongate shaft coupled to the expandable blood conduit; and markings on the elongate shaft configured to convey a distance between a fixed location outside a patient to a target location on the catheter blood pump within the patient.

In one aspect, the catheter blood pump further comprises a processor configured to: receive a first distance from the fixed location to the target location when the distal pressure sensor detects a left ventricular pressure signal; receive a second distance from the fixed location to the target location when the distal pressure sensor or proximal pressure sensor detects an aortic pressure signal; and determine a distance to move the catheter blood pump based on the first distance, the second distance, and the target location.

A non-transitory computing device readable medium having instructions stored thereon is provided, wherein the instructions are executable by a processor to cause a computing device to perform a method comprising: receive a first distance from a fixed location outside a patient to a target location in a heart of the patient when a catheter blood pump pressure sensor detects a left ventricular pressure signal; receive a second distance from the fixed location to the target location when the catheter blood pump pressure sensor detects an aortic pressure signal; and determine a distance to move the catheter blood pump based on the first distance, the second distance, and the target location.

In one aspect, the computer implemented method further includes the step of outputting the distance on a display.

In one aspect, a method of positioning a catheter blood pump is provided, comprising: advancing a catheter blood pump across an aortic valve of a subject; obtaining two or more diagnostic images of the catheter blood pump in two or more imaging planes; calculating a bend angle of the catheter blood pump based on the two or more diagnostic images; and indicating the bend angle to a user of the catheter blood pump.

In some aspects, indicating the bend angle further comprises outputting the bend angle on a display.

In one aspect, the method further comprises identifying a proximal axis of the catheter blood pump and a distal axis of the catheter blood pump.

In some aspects, the proximal axis comprises an impeller axis.

In other aspects, the distal axis comprises an axis of a distal sensor of the catheter blood pump.

In one aspect, the bend angle comprises an angle between the proximal axis and the distal axis.

In some aspects, indicating the bend angle further comprises indicating whether the bend angle exceeds a bend angel threshold.

In one aspect, the method includes instructing the user to reposition the catheter blood pump.

In some aspects, the method includes calculating a distance between the inlet and an annuls of the subject.

In some aspects, the method includes calculating a distance between the outlet and an annuls of the subject.

In other aspects, the method includes indicating to the user an amount to advance the catheter blood pump based on the calculated distance.

These and other details and aspects are described herein.

### BRIEF DESCRIPTION OF THE DRAWINGS

FIG. 1 is a side view of an exemplary blood pump that includes an expandable scaffold that supports a blood conduit with an impeller housed therein.

FIGS. 2A-2E illustrate one embodiment of a sequence of placing a blood pump in a heart of a patient.

FIG. 3 shows catheter blood pump feature locations and distances.

FIG. 4A shows catheter position relative to a fixed location on the patient, such as the introducer sheath.

FIG. 4B shows printed length markings on a shaft of the catheter blood pump.

FIGS. 5A-5D show one method of optimally positioning a catheter blood pump.

FIG. 6 is a flowchart describing an algorithm for positioning a catheter blood pump.

FIGS. 7A-7B show two fluoro views of a blood pump positioned within a heart of a subject.

FIGS. 8A-8C show additional views of a blood pump positioned within the heart of a subject, including techniques for determining a bend angle of the blood pump.

### DETAILED DESCRIPTION

The present disclosure is related to medical devices, systems, and methods of use and manufacture. In particular, described herein are pumps adapted to be disposed within a physiologic vessel, wherein the distal pump portion includes one or more components that act upon fluid. For example, the pumps herein may include one or more rotating members that when rotated, can facilitate the movement of a fluid such as blood.

Any of the disclosure herein relating to an aspect of a system, device, or method of use can be incorporated with any other suitable disclosure herein. For example, a figure describing only one aspect of a device or method can be included with other embodiments even if that is not specifically stated in a description of one or both parts of the disclosure. It is thus understood that combinations of different portions of this disclosure are included herein.

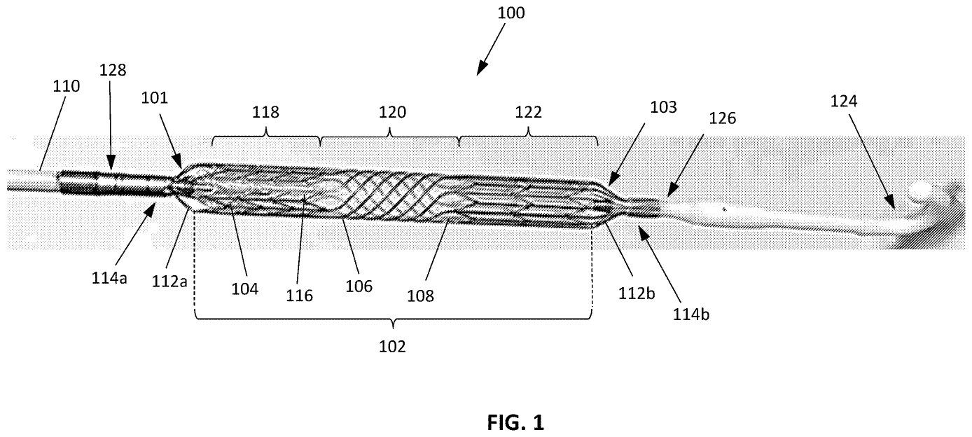

FIG. 1 shows a side view of an exemplary intravascular catheter blood pump 100. The blood pump 100 includes an expandable/collapsible blood conduit 102 that is configured to transition between an expanded state, as shown in FIG. 1, and a collapsed state (not shown). For example, the conduit 102 may be in the collapsed state when confined within a delivery catheter for delivery to the heart, expanded upon release from the delivery catheter for blood pumping, and collapsed back down within the delivery catheter (or other catheter) for removal from heart. When in the expanded state, the conduit 102 is radially expanded so as to form an inner lumen for passing blood therethrough. When in the expanded state, the inner lumen of the conduit 102 may be configured to accommodate blood pumped by one or more impellers therein. The one or more impellers may be collapsible so that they may collapse to a smaller diameter when the conduit 102 is in the collapsed state. The one or more impellers may be positioned within one or more impeller regions of the conduit 102. In some examples, the impeller region(s) of the conduit 102 is/are radially stiffer than other regions (e.g., adjacent regions) of the conduit 102 to prevent the impeller(s) from contacting the interior walls of the conduit 102.

In this example, the blood pump 100 includes an impeller 104 within a proximal portion of the conduit 102. In some cases, the blood pump 100 can include more than one impeller. For example, the blood pump 100 may include a second impeller in a distal region 122 of the fluid conduit 102. In some cases, blood pump 100 may include more than two impellers. The conduit 102 includes a first (e.g., proximal) end having a first (e.g., proximal) opening 101, and a second (e.g., distal) end having a second (e.g., distal) opening 103. The first opening 101 and second opening 103 may be configured as and an inlet and outlet for blood. For example, blood may largely enter the conduit 102 via the second (e.g., distal) opening 103 and exit the conduit 102 via the first (e.g., proximal) opening 101. In such case, the second opening 103 acts as a blood inlet and the first opening 101 acts as a blood outlet. The one or more impellers (e.g., impeller 104) may be configured to pump blood from the inlet toward the outlet. In an exemplary operating position, the second opening 103 (e.g., inlet) may be distal to the aortic valve, in the left ventricle, and the first opening 101 (e.g., outlet) may be proximal to the aortic valve (e.g., in the ascending aorta).

The conduit 102 includes a tubular expandable/collapsible scaffold 106 that provides structural support for a membrane 108 that covers at least a portion of inner surfaces and/or outer surfaces of the scaffold 106. The scaffold 106 includes a material having a pattern of openings with the membrane 108 covering the openings to retain the blood within the lumen of the conduit 102. The scaffold 106 may be unitary and may be made of a single piece of material. For example, the scaffold 106 may be formed by cutting (e.g., laser cutting) a tubular shaped material. Exemplary materials for the scaffold 106 may include one or more of: nitinol, cobalt alloys, and polymers, although other materials may be used.

The blood pump 100 includes proximal struts 112a that extend from the scaffold 106 near the first opening 101 (e.g., blood outlet region) and distal struts 112b that extend from the scaffold 106 near the second opening 103 (e.g., blood inlet region). The proximal struts 112a are coupled to first hub 114a of a proximal shaft 110. The distal struts 112b are coupled to second hub 114b of a distal portion 114. In this example, the first hub 114a includes a bearing assembly through which a central drive cable 116 extends. The drive cable is operationally coupled to and configured to rotate the impeller 104.

In some cases, the impeller 104 is fully positioned axially within the conduit 102. In other cases, a proximal portion of the impeller 104 is positioned at least partially outside of the conduit 102. That is, at least a portion of the impeller may be positioned in axially alignment with a distal portion of the struts 112a.

The conduit 102 and the scaffold 106 may characterized as having a proximal region 118, a central region 120, and a distal region 122. The central region 120 may be configured to be placed across a valve (e.g., aortic valve) such that the proximal region 118 is at least partially within a first heart region (e.g., ascending aorta) and the distal region 122 is at least partially within a second heart region (e.g., left ventricle). The proximal region 118 (and in some cases the distal region 122) may be configured to house an impeller therein. The proximal region 118 may (and in some cases the distal region 122) has a stiffness sufficient to withstand deformation during operation of the blood pump 100 when within the beating heart and to maintain clearance (i.e., a gap) between an impeller region of the blood pump 100 and the rotating impeller 104. The distal region 122 includes the second (e.g., distal) opening 103 of the conduit 102, and may serve as the blood inlet for the conduit 102.

The central region 120 may be less rigid relative to the proximal region 118 (and in some cases the distal region 122). The higher flexibility of the central region 120 may allow the central region 120 to deflect when a lateral force is applied on a side of the conduit 102, for example, as the conduit 102 traverses through the patient's blood vessels and/or within the heart. For example, the central region 120 may be configured to laterally bend upon a lateral force applied to the distal region 122 and/or the proximal region 118. In some cases, it may be desirable for the central region 120 to laterally bend as the conduit 102 traverses the ascending aorta and temporarily assume a bent configuration when the conduit 102 is positioned across an aortic valve. In this example, the central region 120 includes a helical arrangement of longitudinally running elongate elements configured to provide flexibility for lateral bending. In some examples, a distal tip 124 of the blood pump 100 is curved to form an atraumatic tip. In some cases, the distal tip 124 flexible (e.g., laterally bendable) to enhance the atraumatic aspects of the distal tip 124. For example, the distal tip 124 may be sufficiently flexible to bend when pressed against tissue (e.g., by a predetermined amount of force) to prevent puncture of the tissue.

The first hub 114a (e.g., proximal hub) and/or the second hub 114b (e.g., distal hub) may include features that promote smooth blood flow into and/or out of the conduit 102. Such features may prevent or reduce the occurrence of stagnant and/or turbulent blood flow that may otherwise tend to occur in regions near the first opening 101 (e.g., outlet region) and/or the second opening 103 (e.g., inlet region) of the conduit 102. Since stagnant and/or turbulent blood flow is associated with blood coagulation and/or clotting, measures to reduce this can be beneficial to patient outcomes.

The blood pump 100 can include one or more pressure sensors configured to take a pressure reading inside the patient. In some embodiments, the blood pump 100 can include a distal pressure sensor 126 positioned near or adjacent to the inlet or distal hub 114b, and a proximal pressure sensor 128 positioned near or adjacent to the outlet or proximal hub 114a. The pressure sensors can be any pressure sensor known in the art. In some embodiments, the pressure sensors can comprises MEMs pressure sensors.

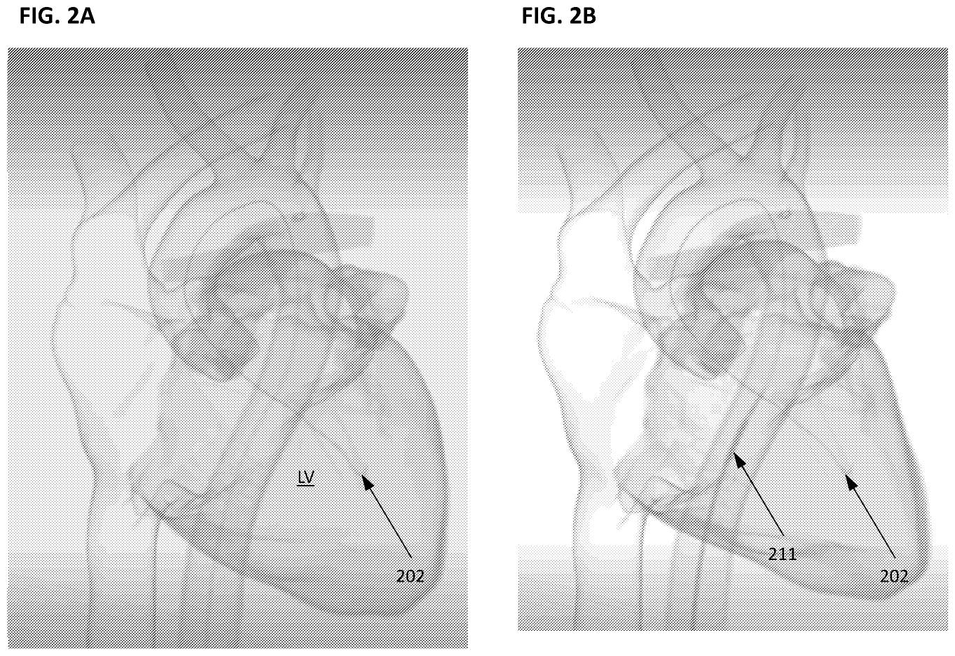

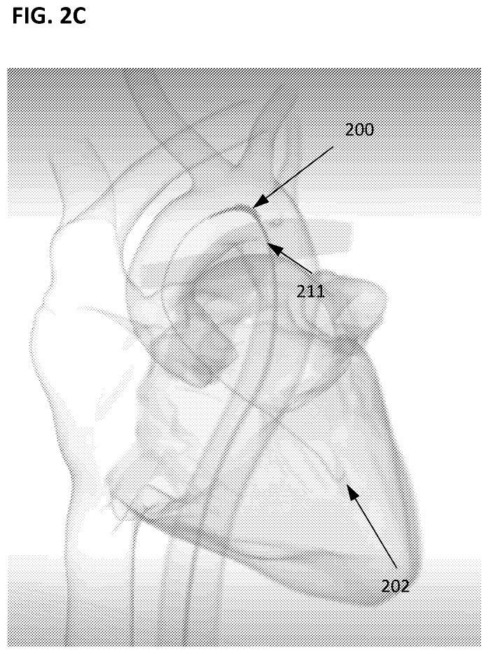

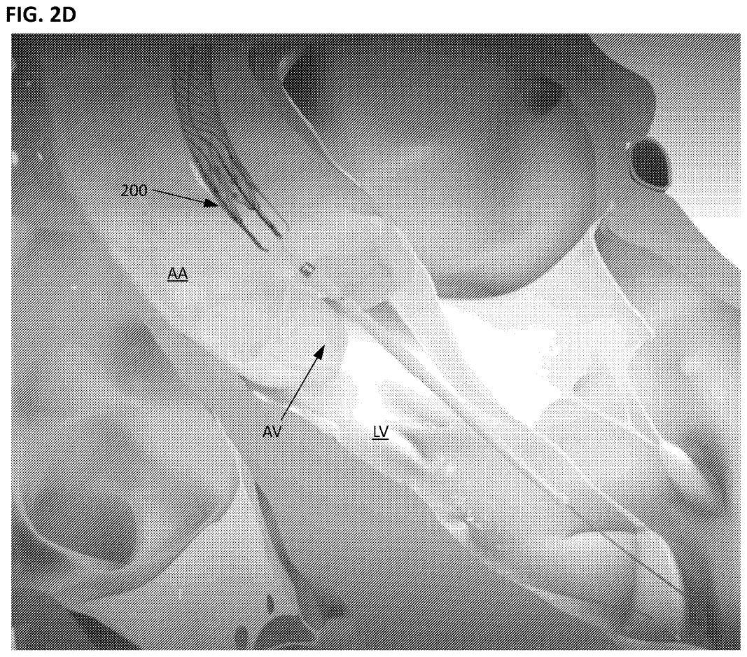

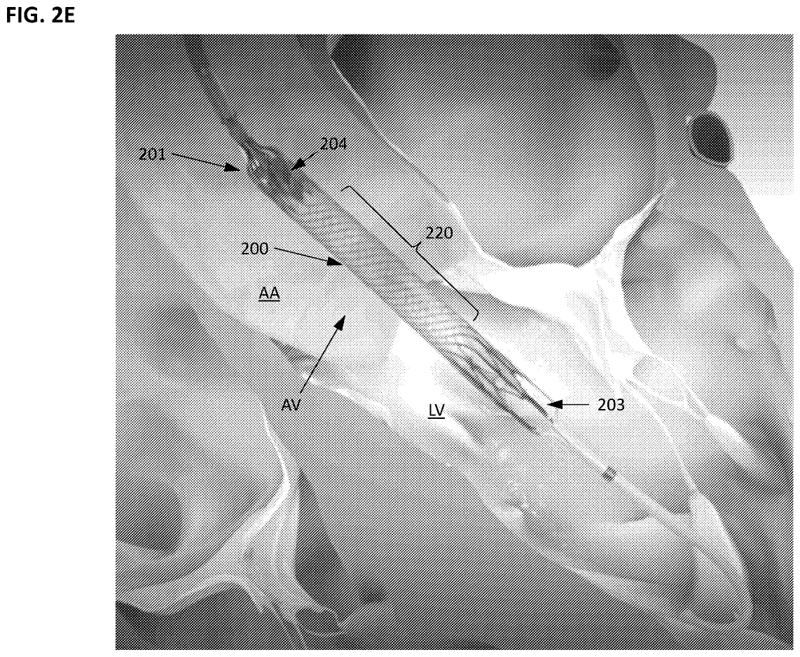

Methods of delivering and placing the blood pump will now be discussed. First, access to the patient's vasculature can be obtained (e.g., without limitation, via femoral access) using an appropriately sized introducer sheath. Using standard valve crossing techniques, in FIG. 2A a guide wire 202 may be advanced until it is positioned securely in the target location (e.g., left ventricle “LV”). The guide wire positioning can be performed under imaging such as fluoroscopy

Next, referring to FIG. 2B, the blood pump, positioned in a compressed or delivery configuration within an introducer sheath 211, can be advanced over the guide wire towards a target location.

In FIG. 2C, the introducer sheath 211 can be retracted, exposing the pump portion and causing the pump to expand from the compressed or delivery configuration to an expanded configuration (e.g., the configuration as shown in FIG. 1). In some examples, the pump can be expanded in the aorta near the bifurcation to the iliac arteries. The introducer sheath can be long enough to extend to the aorta to allow for expansion of the pump in the aorta. Conventional introducer sheaths are not long enough to enable unsheathing of a pump in this location.

In FIG. 2D, the expanded blood pump 200 can be advanced over the guidewire 202, first into the ascending aorta (“AA”), and then in FIG. 2E, to the target location in which the blood pump extends across the aortic valve (“AV”) and into the left ventricle (“LV”). In FIG. 2E, the blood pump inlet 203 is positioned in the LV and the blood pump outlet 201 and proximal impeller 204 are positioned in the ascending aorta AA. The central region 220 of the blood pump, which may be more flexible than the impeller regions, as described in more detail above, spans the aortic valve AV.

Techniques for accurately and properly positioning the blood pump at a target location within the patient will now be described. In one embodiment, it is desired to maintain the pump in proper position across the aortic valve (AV) with the distal inlet within the left ventricle (LV) and the proximal outlet in the ascending aorta (AA). This positioning can be essential for proper pump function and minimization of potential complications. The techniques described herein do not rely on fluoroscopic imaging and could be performed by the bedside in the ICU, an ambulance, etc. In some embodiments, the methods and techniques described herein rely on known distances known to the end user or the system. The known distances can be provided, for example, in the blood pump's instructions for use (IFU), a separate datasheet, stored in memory of the console or computing system of the blood pump, or on a separate electronic device such as a PC, smartphone, etc.

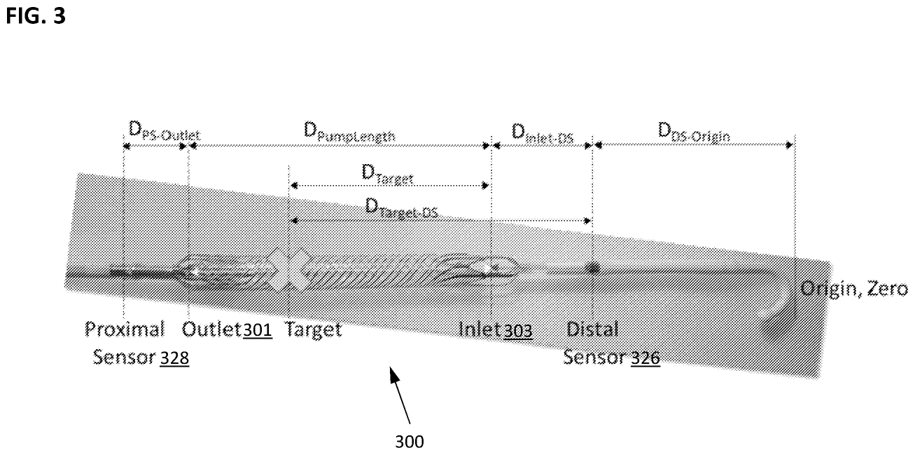

FIG. 3 shows a blood pump 300, such as any blood pump described herein, including several pump features and locations previously described. As shown, the pump can include an inlet 303 and an outlet 301, distal pressure sensor 326 and proximal pressure sensors 328, and a targeted location “Target” that is a distance D_{Target} along the pump measured from the inlet 303 to where the aortic valve should be centered when the pump is optimally placed or positioned within the anatomy. The locations and distances shown in FIG. 3 are described below:
a. D_{DS-Origin}, is the distance from the distal-most end of the catheter and the distal pressure sensor 326.
b. D_{Inlet-DS}, is the distance between the inlet 303 and the distal pressure sensor 326.
c. D_{PumpLength}, is the distance between the pump inlet 303 and pump outlet 301.
d. D_{PS-Outlet}, is the distance between the pump outlet 301 and the proximal pressure sensor 328.
e. D_{Target}, is the distance to the Target location along the pump length measured from the pump inlet 303 to the Target location along the pump where the aortic valve should be centered when optimally positioned. The Target location depends on a number of factors including pump length, proximal impeller location, proximal impeller size, etc. Depending on these dimensions, the Target location can be, for example, D_{Target}=⅔×D_{PumpLength}, D_{Target}=½×D_{PumpLength}, D_{Target}=⅓×D_{PumpLength}, etc. It should be understood, however, that the Target location is distal to the location of the proximal impeller of the blood pump. In some embodiments, the Target location is within the central portion of the blood pump, which can be more flexible than other sections of the blood pump. As described above, the blood pump of the present disclosure is proximally located near the outlet of the pump, and the impeller and outlet are intended to be placed in the AA while the inlet is intended to be placed in the LV. The Target location is the position of the pump that is intended to be placed at the AV to enable optimal placement of the inlet and outlet in the aforementioned locations.
f. D_{Target-DS}, is the distance from the Target location to the distal sensor 326; equal to D_{Inlet-DS}+D_{Target}.

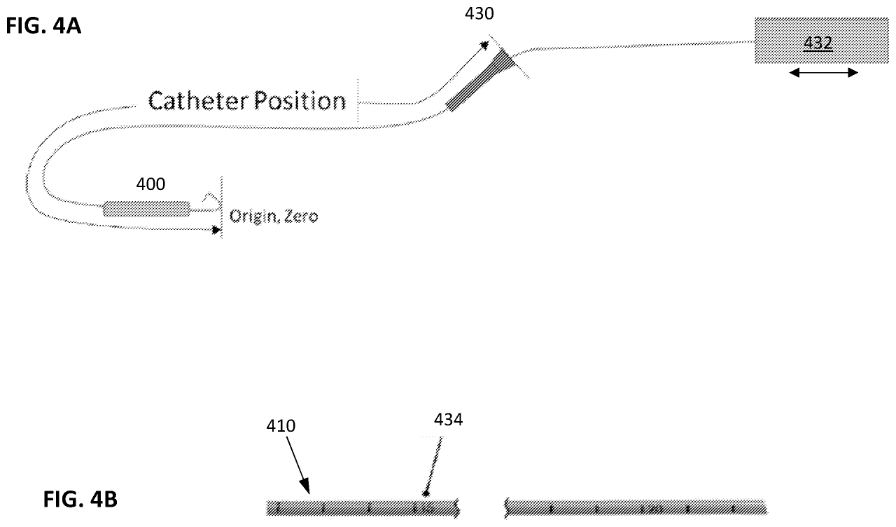

Additionally, the catheter position, or relative position of the blood pump 400 to a fixed location on the patient can be known, such as the distance between the distal end of the pump, Origin, Zero, and a position along a secured introducer sheath 430, as shown in FIG. 4A. This distance represents the length of the blood pump catheter between the introducer sheath (or other fixed location) and the end of the pump. The distance is defined between the pump distal end and a fixed location since a location of handle 432 of the pump can be moved back and forth during a positioning procedure, while the secured introducer sheath 430 remains in a constant position (e.g., attached to the patient's skin).

In some embodiments, this distance from the fixed location (e.g., introducer sheath 430) to the distal end of the blood pump can be provided by printed length markings along the length of the catheter shaft. FIG. 4B shows an example of printed length markers 434 along the catheter shaft 410 of the blood pump. In some embodiments, these length markings could incorporate encoding (e.g., QR code) that could be read by an imaging sensor or characters that could be read by optical character recognition (OCR). The imaging sensor or optical character recognition device could be placed, for example, at the fixed location on the patient such as at the introducer sheath.

The catheter position information can be recorded or documented in a number of ways, including: 1) mentally recording or remembering the length of catheter inserted into the patient, 2) manually recording the length of catheter inserted into the patient (e.g., pen and paper), 3) Manually inputting the length of catheter inserted into the patient (e.g., by typing or inputting the information into the blood pump console or another computing device such as a smartphone, tablet, or PC), or 4) automatically inputting the catheter position information into the console, PC, smartphone, or tablet. In some embodiments, the automatic input could come from digitally connected devices such as imaging sensors or OCR scanners which can be electronically coupled or connected to the device console, PC, tablet, or smartphone. These sensors could, for example, scan or read markings on the catheter shaft as described above to identify the distance from the fixed location or sensor to the distal end of the catheter (or any other known position on the catheter relative to the markings).

Algorithms, methods, and techniques for utilizing pressure sensor measurements to determine optimal pump placement are now discussed. The algorithms, methods, and techniques can be implemented in a processor of the catheter blood pump, such as in a processor of a console or computing system electrically coupled to the catheter blood pump. The console can include, for example, a non-transitory computing device readable medium having instructions stored thereon, wherein the instructions are executable by a processor to cause a computing device to perform the algorithms, methods, and techniques discussed herein.

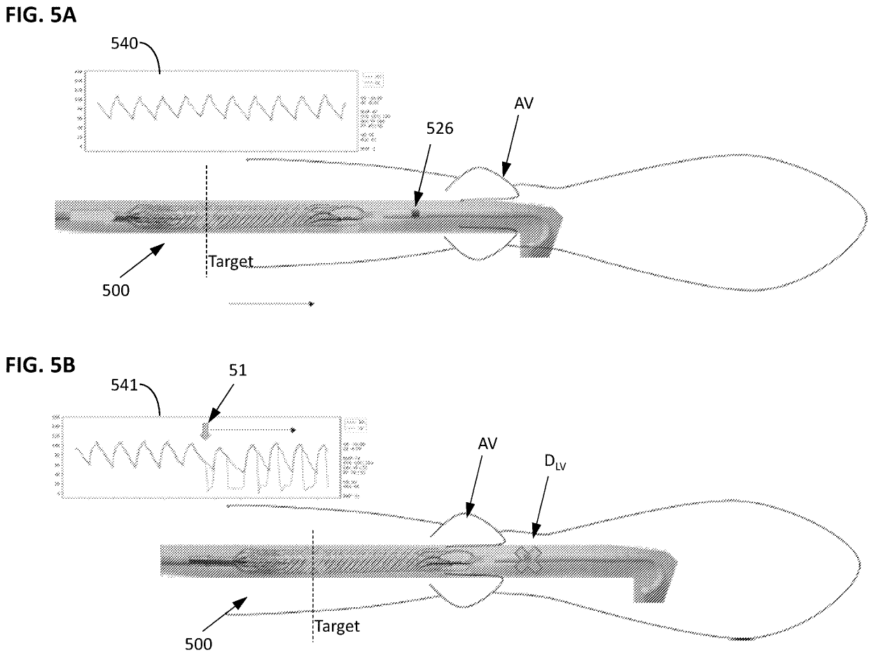

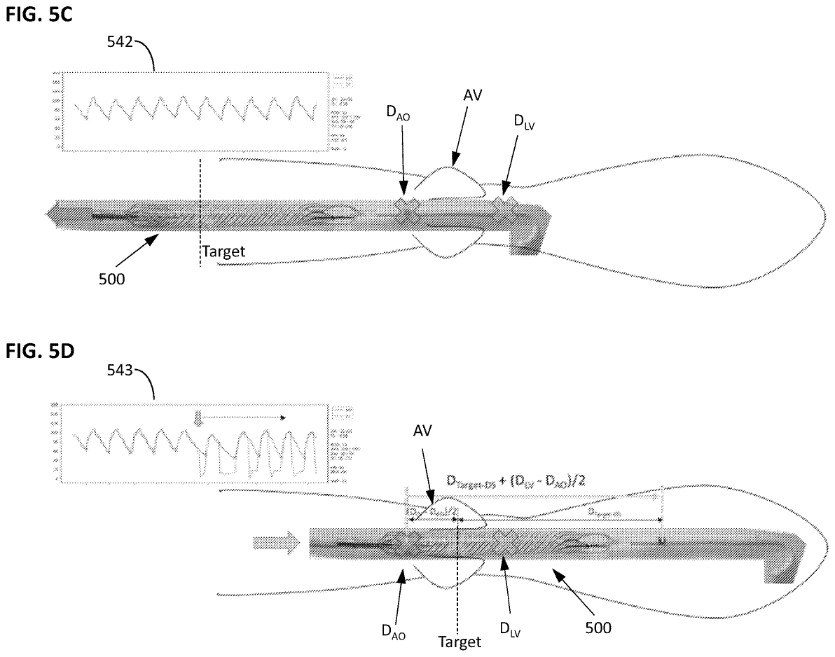

FIG. 5A shows the blood pump 500 being slowly advanced across the aortic valve (AV) of the patient, with plot 540 showing a pressure waveform recorded by the distal pressure sensor 526. In FIG. 5B, the distal pressure sensor 526 has been advanced across the aortic valve AV. Plot 541 in FIG. 5B shows the change in pressure waveform at arrow 51 where the distal pressure sensor 526 begins to detect a left ventricular pressure signal. Although the distal pressure sensor 526 is in the left ventricle in FIG. 5B, the inlet of the blood pump is still at least partially blocked by the aortic valve, so the blood pump is not yet in an ideal position.

When the distal pressure sensor begins to detect the LV signal, advancement of the blood pump can be stopped. In some examples, the catheter position (e.g., the length of catheter inserted into the patient relative to a fixed location) can be recorded (e.g., the marker reading on the catheter at the introducer sheath). This catheter position can be recorded as D_{LV}, e.g., the distance from the introducer sheath to the left ventricle. In some aspects, the system and/or processor can automatically record the D_{LV} catheter position when the left ventricular pressure signal is detected. In another embodiment, a user of the device or system can provide an input to record the D_{LV} catheter position, e.g., by pressing a button on the handle or on the console of the blood pump or system.

Next, referring to FIG. 5C, the catheter can be pulled back to move the distal pressure sensor back into the aorta. When the distal pressure sensor detects an aortic pressure signal, as shown in plot 542, the catheter position can again be marked or recorded as D_{AO}, the distance from the introducer sheath (or other fixed location) to the aortic valve on the aortic side. In some aspects, the system and/or processor can automatically record the D_{AO} catheter position when the left ventricular pressure signal is detected. In another embodiment, a user of the device or system can provide an input to record the D_{AO} catheter position, e.g., by pressing a button on the handle or on the console of the blood pump or system.

Next, referring to FIG. 5D, the distal pressure sensor can again be advanced into the ventricle by the distance D_{Target-DS}+(D_{LV}−D_{AO})/2, where D_{Target-DS} is the known distance based on the device being used, and where D_{LV} and D_{AO} were obtained in the prior steps shown in FIGS. 5B and 5C. The arrow in plot 543 in FIG. 5D shows the change in pressure waveform when the distal pressure sensor 526 begins to detect a left ventricular pressure signal. This technique and algorithm places the blood pump in the Target location, which is a distance D_{Target} from the inlet of the blood pump to the Target location, approximately midway in the aortic valve region.

Alternate positioning algorithms are provided with different combinations of known sensor location(s) relative to the aortic valve to position the pump relative to the aortic valve region. In a first alternative, the proximal sensor of the blood pump could alternatively be used in a manner similar to described above, instead of the distal sensor, with the disadvantage of having to insert more of the catheter into the ventricle to get the appropriate pressure readings. All of the steps discussed above in FIGS. 5A-5D could be performed with the proximal sensor as the reference sensor instead of the distal sensor.

In another embodiment, both the distal and proximal sensors could be used for pump positioning, where the pump is fully advanced into the ventricle and the difference between catheter positions when the distal sensor first detected the left ventricular pressure signal and when the proximal sensor first detected the left ventricular pressure signal, along with the known physical distance between the sensors, could be used to determine how much to pull the catheter back towards the ventricle in order to place the target in the aortic valve region. Again, this disadvantage here, compared to the technique described above in FIGS. 5A-5D, is having to insert more of the catheter blood pump into the ventricle since the proximal pressure sensor must be inserted into the left ventricle to measure a ventricular pressure signal.

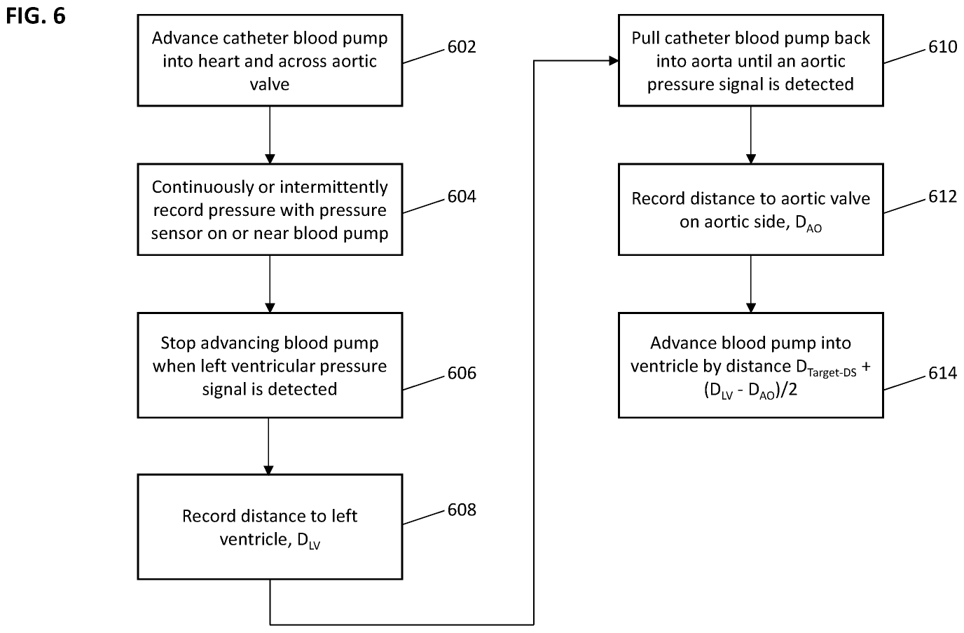

FIG. 6 is a flowchart describing the method steps associated with FIGS. 5A-5D and described above. At step 602, the catheter blood pump can be advanced into the heart and across the aortic valve. At step 604, the blood pump pressure sensor (e.g., the distal pressure sensor, or alternatively, the proximal pressure sensor) can continuously or intermittently record pressure signals in the heart. At step 606, the method can include stopping advancement of the catheter blood pump when a left ventricular pressure signal is detected. At step 608, the distance to the left ventricle, D_{LV}, can be recorded. At step 610, the catheter blood pump can be pulled back into the aorta until an aortic pressure signal is detected. At step 612, the distance to the aortic valve on the aortic side of the heart, D_{AO}, can be recorded. Finally, at step 614, the catheter blood pump can be advanced by a distance equal to D_{Target-DS}+(D_{LV}−D_{AO})/2. As described above, D_{Target-DS} is the distance from D_{Target}, the targeted location, to the distal pressure sensor.

Any of the blood pumps described herein may include surfaces with one or more anticoagulant agents. For example, at least a portion of one or more of the hubs, conduits (e.g., scaffold and/or membrane), struts (e.g., proximal and/or distal struts), distal tips and/or impellers of the blood pumps described herein may include a coating or material having an anticoagulant agent. In some cases, the anticoagulant agents may include drugs such as heparin, warfarin and/or prostaglandins.

### Computing Pump Bend Angle to Determine Proper Placement

In other implementations, systems, methods, and algorithms are provided for determining or calculating a bend angle of the blood pump to confirm or determine if the blood pump is properly placed in the anatomy. In some examples, the bend angle can be used to inform the user/physician on next steps required for proper pump placement or positioning.

In one example, the blood pump can be inserted into a subject and placed according to any of the methods or techniques described herein. As described above, optimal blood pump placement comprises placing the inlet of the blood pump in the LV and the blood pump outlet and proximal impeller in the ascending aorta AA, with the central region of the blood pump, which may be more flexible than the impeller regions, spanning the aortic valve AV.

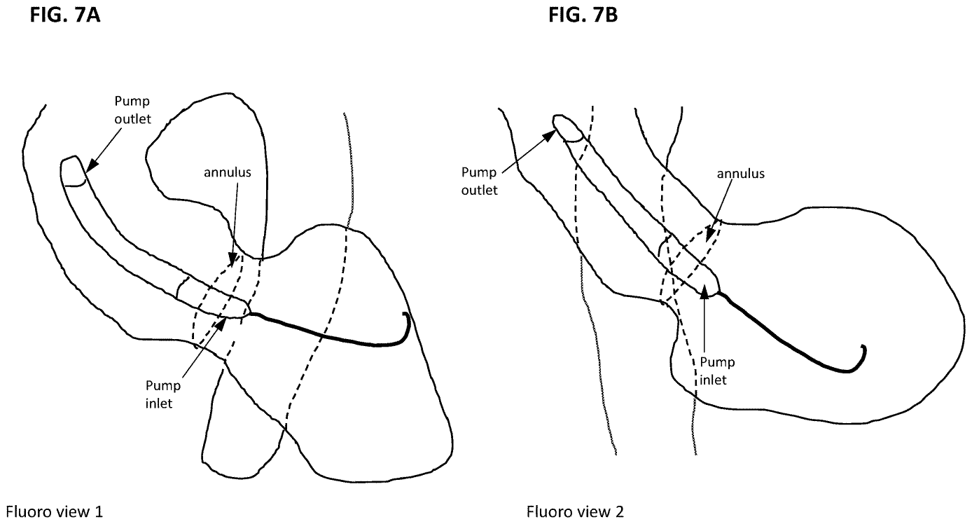

Once the user/physician believes that the pump is properly placed, placement can be confirmed with fluoroscopic (“fluoro”) imaging. However, since patient anatomy can vary from patient to patient, including ventricular volume, arch radius, and orientation of the heart anatomy within the patient, fluoro images in a single plane may not accurately capture aspects of the blood pump that could otherwise be indicators on whether or not the pump is in the proper position. For example, if a blood pump is bent or kinked in a direction predominantly out of plane from the fluoro images, the bend or kink may not be apparent on the imaging. Therefore, referring to FIGS. 7A-7B, a first aspect of the present disclosure includes taking fluoro images in at least two different planes. In some embodiments, the two or more fluoro images can be taken at times corresponding to min/max anatomical movement (e.g., systole/diastole).

FIG. 7A shows a fluoro image of blood pump placed in a subject's heart in a first fluoro view, with the patient's left ventricular & aortic lumens overlayed, and FIG. 7B shows a fluoro image of the same blood pump placement in the subject's heart in a second fluoro view with the patient's left ventricular & aortic lumens overlayed. As shown in FIGS. 7A-7B, the first fluoro view shows a tight bend towards the proximal end of the pump, while the second view, in which the bend is mostly out of plane, shows a relatively straight pump (or a pump with only a slight bend or curvature). For example, the first fluoro view may be taken at LAO 5° (left anterior oblique), CRA 35° (cranial), and the second fluoro view may be taken at RAO 25° (right anterior oblique), CAU 25° (caudal). In general, it is desirable to space apart the fluoro views by a sufficient angle such that any pump bending or kinking may be seen in at least one of the views. In some aspects of the disclosure, the fluoro views can be spaced apart by approximately 60-90 degrees between the first view and the second view. In the provided example, there is a 30 degree difference between the LAO and RAO angles and 60 degree difference between the CRA and CAU angles. While the example of FIGS. 7A-7B shows only two different fluoro views, it should be understood that more than two fluoro views can further inform the user/physician on pump placement and/or pump bending.

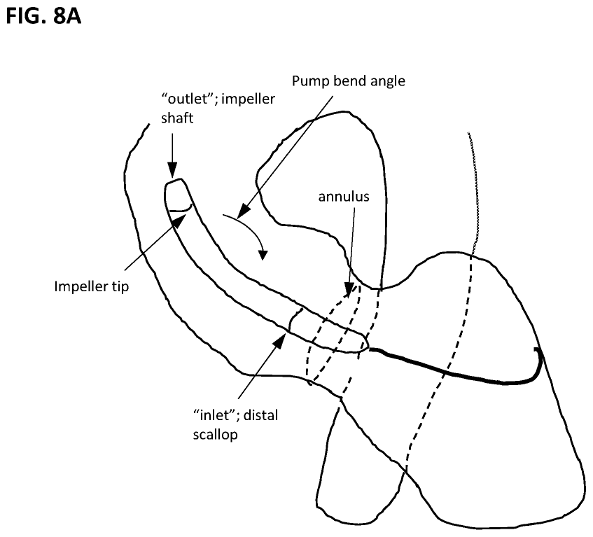

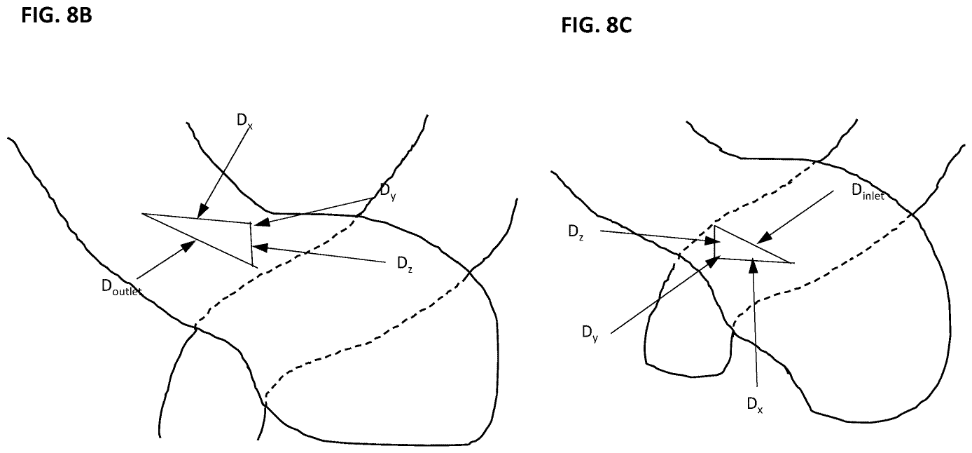

Tight bends or kinks in the pump can be an indication that the pump is not optimally placed. For example, a tight bend or kink may inhibit the pump from being fully advanced into the optimal position with the inlet in the LV and the outlet in the AA. In the example of FIG. 8A, the inlet of the pump is in the vicinity of the aortic valve annulus instead of being fully advanced into the LV, which can inhibit optimal pump performance. This disclosure therefore provides techniques for identifying situations in which the pump is not optimally placed so as to inform the physician on steps that can be taken to correct placement of the pump. Referring to FIG. 8A, multiple measurements from the fluor images can be taken, including the distance from the AV annulus to the Outlet/impeller shaft; the distance from the AV annulus to the inlet/distal scallop; and/or the bend angle defined by the axis of the outlet/impeller shaft to the axis if the distal pressure senor.

In one aspect, referring to FIGS. 8B and 8C, the location of relevant anatomical landmark(s) for positioning the pump, such as the location of the aortic valve or annulus, can be determined through various means such as placing a guidewire or guide catheter against the aortic valve, taking an aortogram, overlaying data from another imaging modality (e.g., computed tomography, echocardiography, etc.). The pump is then advanced across the aortic valve and the two or more aforementioned fluoro views are captured. The anatomical landmark(s) can then be re-referenced in each view and the distance and/or angles from the anatomical landmark(s) to positioning landmarks on the pump (e.g., Dₒᵤₜₗₑₜ, Dᵢₙₗₑₜ, Dₓ, D_{y}, D_{z}, radiopaque markers) and/or distance and/or angles from two or more different positioning landmarks (e.g., angle between the pump outlet and pump inlet) can be measured. Target value ranges for these distances can be provided by the pump manufacturer and included in the IFU, displayed on the console, or otherwise conveyed to the pump operator. If these measured pump positioning distances/angles are outside the prescribed range(s), the operator can then choose at their discretion to attempt to reposition the pump into a more optimal orientation.

1. A method of positioning a catheter blood pump, comprising:
advancing a catheter blood pump across an aortic valve of a subject;
continuously or intermittently recording pressure signals in the heart with a pressure sensor of the catheter blood pump;
stopping advancement of the catheter blood pump when a left ventricular pressure signal is detected with the pressure sensor;
recording a first distance from a fixed location on, in, or outside the subject to a left ventricle, D_{LV};
pulling back the catheter blood pump into the aorta until an aortic pressure signal is detected with the pressure sensor;
recording a second distance from the fixed location to the aortic valve, D_{AO};
determining a target distance to advance the catheter blood pump based on the first distance, the second distance, and catheter blood pump dimensions; and
advancing the catheter blood pump by the target distance to optimally position the pump.

2. The method of claim 1, wherein the pressure sensor is positioned near a distal end of the catheter blood pump.

3. The method of claim 1, wherein the pressure sensor is positioned near a proximal end of the catheter blood pump.

4. The method of claim 1, wherein the catheter blood pump is optimally positioned with an inlet in the ventricle and an outlet in the aorta.

5. The method of claim 4, wherein an impeller of the catheter blood pump is positioned in the aorta.

6. The method of claim 1, wherein determining the target distance comprises solving for D_{Target-DS}+(D_{LV}−D_{AO})/2, where D_{Target-DS} is the distance from D_{Target}, the targeted location, to the pressure sensor.

7. A method of positioning a catheter blood pump, comprising:
advancing a catheter blood pump across an aortic valve of a subject;
continuously or intermittently recording pressure signals in the heart with a distal pressure sensor and a proximal pressure sensor of the catheter blood pump;
recording a first distance from a fixed location on, in, or outside the subject to a left ventricle, D_{LV}, when a first left ventricular pressure signal is detected with the distal pressure sensor;
recording a second distance from a fixed location on, in, or outside the subject to the left ventricle, D_{LV}, when a second left ventricular pressure signal is detected with the proximal pressure sensor;
determining a target distance to pull back the catheter blood pump based on the first distance, the second distance, and catheter blood pump dimensions; and
pulling back the catheter blood pump by the target distance to optimally position the pump.

8. A catheter blood pump, comprising:
an expandable blood conduit defining a lumen between an inlet and an outlet;
an impeller at least partially disposed within the blood conduit;
a proximal pressure sensor positioned proximally from the outlet;
a distal pressure sensor positioned distally from the inlet;
an elongate shaft coupled to the expandable blood conduit;
markings on the elongate shaft configured to convey a distance between a fixed location outside a patient to a target location on the catheter blood pump within the patient; and
a processor configured to:
receive a first distance from the fixed location to the target location when the distal pressure sensor detects a left ventricular pressure signal;
receive a second distance from the fixed location to the target location when the distal pressure sensor or proximal pressure sensor detects an aortic pressure signal; and
determine a distance to move the catheter blood pump based on the first distance, the second distance, and the target location.

9. (canceled)

10. A non-transitory computing device readable medium having instructions stored thereon, wherein the instructions are executable by a processor to cause a computing device to perform a method comprising:
receive a first distance from a fixed location outside a patient to a target location in a heart of the patient when a catheter blood pump pressure sensor detects a left ventricular pressure signal;
receive a second distance from the fixed location to the target location when the catheter blood pump pressure sensor detects an aortic pressure signal; and
determine a distance to move the catheter blood pump based on the first distance, the second distance, and the target location.

11. The computing device of claim 10, further comprising:
output the distance on a display.

12. A method of positioning a catheter blood pump, comprising:
advancing a catheter blood pump across an aortic valve of a subject;
obtaining two or more diagnostic images of the catheter blood pump in two or more imaging planes;
calculating a bend angle of the catheter blood pump based on the two or more diagnostic images; and
indicating the bend angle to a user of the catheter blood pump.

13. The method of claim 12, wherein indicating the bend angle further comprises outputting the bend angle on a display.

14. The method of claim 12, further comprising identifying a proximal axis of the catheter blood pump and a distal axis of the catheter blood pump.

15. The method of claim 14, wherein the proximal axis comprises an impeller axis.

16. The method of claim 14, wherein the distal axis comprises an axis of a distal sensor of the catheter blood pump.

17. The method of claim 14, wherein the bend angle comprises an angle between the proximal axis and the distal axis.

18. The method of claim 12, wherein indicating the bend angle further comprises indicating whether the bend angle exceeds a bend angel threshold.

19. The method of claim 18, further comprising instructing the user to reposition the catheter blood pump.

20. The method of claim 12, further comprising calculating a distance between the inlet and an annuls of the subject.

21. The method of claim 12, further comprising calculating a distance between the outlet and an annuls of the subject.

22. The method of claim 20, further comprising indicating to the user an amount to advance the catheter blood pump based on the calculated distance.
